(19) United States
(12) Patent Application Publication
(10) Pub. No.: US 2021/0236395 A1
(43) Pub. Date: Aug. 5, 2021

(54) ENCAPSULATED LIQUID COMPOSITION

(71) Applicant: The Procter & Gamble Company, Cincinnati, OH (US)

(72) Inventors: Shufen FAN, Singapore (SG); Kenneth Shun Qiang ANG, Singapore (SG); YunQin LEE, Singapore (SG)

(21) Appl. No.: 17/239,809

(22) Filed: Apr. 26, 2021

Related U.S. Application Data
(63) Continuation of application No. PCT/US2019/057749, filed on Oct. 24, 2019.
(60) Provisional application No. 62/751,711, filed on Oct. 29, 2018.
(60) Provisional application No. 62/790,469, filed on Jan. 10, 2019.
(60) Provisional application No. 62/807,762, filed on Feb. 20, 2019.

(51) Int. Cl. A61K 8/11; A61Q 5/02; A61Q 5/12; A61K 8/02; A61K 8/73

(52) U.S. Cl. CPC A61K 8/11; A61Q 5/02; A61K 2800/42; A61K 8/0245; A61K 8/733; A61Q 5/12

(57) ABSTRACT

Disclosed is an encapsulated liquid composition comprising a shell and the liquid composition contained inside of the shell, wherein the encapsulated liquid composition meets at least one of the conditions: the encapsulated liquid composition has other shapes than spherical shape; the encapsulated liquid composition contains 2 liquid compositions having different colors inside of the shell, and the 2 compositions form stripe, marble, and/or rainbow; and/or the encapsulated liquid composition contains smaller inner capsule, where in the smaller inner capsule is also an encapsulated liquid composition comprising a shell and the liquid composition contained inside of the shell, wherein the shell is formed by a material selected from the group consisting of alginate gel, carrageenan gel, chitosan gel, or mixtures thereof.

### FIELD OF THE INVENTION

The present invention relates to an encapsulated liquid composition comprising a shell and the liquid composition contained inside of the shell, wherein the encapsulated liquid composition meets at least one of the conditions: the encapsulated liquid composition has other shapes than spherical shape; the encapsulated liquid composition contains 2 liquid compositions having different colors inside of the shell, and the 2 compositions form stripe, marble, and/or rainbow; and/or the encapsulated liquid composition contains smaller inner capsule, where in the smaller inner capsule is also an encapsulated liquid composition comprising an alginate gel shell and the liquid composition contained inside of the shell, wherein the shell is formed by a material selected from the group consisting of alginate gel, carrageenan gel, chitosan gel, or mixtures thereof. The present invention provides, for example, aesthetic improvement for the encapsulated liquid compositions having low viscosity. When having the smaller inner capsule, the encapsulated liquid composition may provide one of the followings: such aesthetic improvement; stability improvement; and/or delayed delivery of the benefits of the composition in the inner capsule.

### BACKGROUND OF THE INVENTION

Calcium alginate capsules have been used in a wide range of industries such as cosmetics and foods with a variety of sizes.

For example, Brian G. Zukas et.al discloses the capsules with about 10 mm diameter, in their articles titled “Improved Water Barrier Properties of Calcium Alginate Capsules Modified by Silicone Oil” (Gels 2016, 2(2), 14; doi:10.3390/gels2020014).

Another example could be U.S. Pat. No. 4,765,984, which discloses the use of the alginate/calcium gelation reaction in the formation of single unit dose dental products.

One more example could be from MAILONLINE web magazine article titled “Will this be the end of plastic bottles? Edible water ‘balloon’ that looks like a breast implant will go on sale within a year” (PUBLISHED: 16:13 BST, 11 Apr. 2017 I UPDATED: 16:23 BST, 11 Apr. 2017). This article introduces seaweed spheres created using sodium alginate and calcium chloride, which contains nine ounces (250 ml) of water.

However, in such capsules, there still remains a need to provides at least one of the following:
Providing an encapsulated liquid composition in other shapes than spherical shape, for the liquid composition with a lower viscosity, with alginate gel shell;
Providing an encapsulated liquid composition with a variety of aesthetics, for example: multi-color such as phased, stripe, marble, and rainbow with low viscosity liquid composition;
“Capsule(s) in capsule” which may be preferred for at least one of the followings:
Aesthetic improvement, for example, such that the smaller inner colored capsules are dispersed in the liquid composition in a bigger outer capsule;
Stability improvement, for example, such that a smaller inner capsule can contain material which is immiscible, incompatible and/or reactive with material in the liquid composition outside of the smaller inner capsule but inside of the bigger outer capsule, including, but not limited to gaseous material; and/or
Delayed delivery of the benefits of the composition in the inner capsule.
None of the existing art provides all of the advantages and benefits of the present invention.

### SUMMARY OF THE INVENTION

The present invention is to an encapsulated liquid composition comprising a shell and the liquid composition contained inside of the shell,

wherein the encapsulated liquid composition meets at least one of the conditions: the encapsulated liquid composition has other shapes than spherical shape (hereinafter “shaped embodiment”);
the encapsulated liquid composition contains 2 liquid compositions having different colors in side of the gel shell, and the 2 compositions form stripe, marble, and/or rainbow (hereinafter “multi-color embodiment”); and/or
the encapsulated liquid composition contains smaller inner capsule, where in the smaller inner capsule is also an encapsulated liquid composition comprising a shell and the liquid composition contained inside of the shell (hereinafter “capsule-in-capsule” embodiment), and
wherein the shell is formed by a material selected from the group consisting of alginate gel, carrageenan gel, chitosan gel, or mixtures thereof.

The present invention provides, for example, aesthetic improvement for alginate gel shell encapsulated liquid compositions having low viscosity. In capsule-in-capsule embodiment, the encapsulated liquid composition may provide one of the followings: such aesthetic improvement; stability improvement; and/or delayed delivery of the benefits of the composition in the inner capsule.

None of the existing art provides all of the advantages and benefits of the present invention. These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials. Herein, “mixtures” is meant to include a simple combination of materials and any compounds that may result from their combination.

The term “molecular weight” or “M. Wt.” as used herein refers to the weight average molecular weight unless otherwise stated.

### Shape

In the shaped embodiments, the encapsulated liquid compositions have a shape other than spherical shape. Such other shapes than spherical shapes include, for example, star, heart, flower, triangle, square, cube, pyramid, diamond, rectangular, donut, and oval.

In other embodiments, the encapsulated liquid composition can have any shape including spherical shape and the above shapes.

### Aesthetics/Multi-Color

The encapsulated liquid composition can contain one liquid composition or two or more liquid compositions. When containing two or more liquid compositions especially when such two or more liquid compositions have different colors, it may be preferred that such two or more liquid compositions have certain viscosities or densities to not mixed in the capsule. The liquid composition inside of the shell can have a variety of aesthetics, for example: homogeneous single color; multi-color such as phased, stripe, marble, and rainbow, wherein such multi-color can be made by two or more liquid compositions having different colors, or one liquid composition having different colors; dispersing colored objects such as flower petals and smaller inner capsules with a variety of size, shape and color; changing color depending on environmental matters (such as temperature, pH, pressure, moisture and humidity).

In the multi-color embodiment, the encapsulated liquid composition contains 2 compositions having different colors, and the 2 compositions form stripe, marble, and/or rainbow.

The shell also can have a variety of aesthetics, for example: homogeneous single color with or without containing dyes and/or mica; multi-color such as phased, stripe, marble, and rainbow; changing color depending on environmental matters (such as temperature, pH, pressure, moisture and humidity).

### Capsule in Capsule

In the capsule-in-capsule embodiment, the encapsulated liquid composition (outer capsule) has an outer capsule shell, and contains 1^{st} liquid composition and smaller inner capsule(s) inside of the outer capsule shell, wherein the smaller inner capsule is also an encapsulated liquid composition comprising an inner capsule shell and 2^{nd} liquid composition contained inside of the inner capsule shell. The 1^{st} and 2^{nd} compositions can be same or different.

This capsule-in-capsule embodiment may be preferred especially for:
Aesthetic improvement, for example, such that the smaller inner colored capsules are dispersed in the liquid composition in a bigger outer capsule; and/or
Stability improvement, for example, such that a smaller inner capsule can contain material which is immiscible, incompatible and/or reactive with material in the liquid composition outside of the smaller inner capsule but inside of the bigger outer capsule, including, but not limited to gaseous material.
Delayed delivery of the benefits of the liquid hair care composition inside of the inner capsule.

### Size

The encapsulated liquid composition may have a diameter of at least about 100 nanometer in diameter and no upper limitation, preferably from about 0.5 micrometer to about 150 mm, more preferably from about 5 mm to about 150 mm, still more preferably from about 5 mm to about 100 mm, furthermore preferably from about 5 mm to about 60 mm.

### Liquid Composition

Liquid compositions useful herein can be anhydrous liquid composition or aqueous liquid composition. The liquid composition has a viscosity of from about 1 cp to about 1,000,000 cp, more preferably from about 1 cp to about 500,000 cp, still more preferably from about 1 cp to about 200,000 cp.

The liquid composition can be anything, for example, personal care composition, and non-personal care compositions such as fabric care, home care, air care and/or dish care compositions.

The liquid composition useful herein can be personal care composition which is selected from the group consisting of a hair care composition, a body care composition, a facial skin care composition, shave care, oral care compositions, personal health care, and mixtures thereof, and preferably is a hair care composition such as shampoo, conditioner, treatment, styling, growth and colorant

The liquid composition useful herein preferably has a pH of from about 3 to 9, more preferably from about 3.5 to about 7.5

### Shell Material

The shell is formed by a material selected from the group consisting of alginate gel, carrageenan gel, chitosan gel, or mixtures thereof, preferably alginate gel. These gels are usually formed by ionic crosslinking reaction between a polymer such as alginate, carrageenan and chitosan, and ions or source of ions.

### Alginate Gel Shell

The alginate gel shell is usually formed by crosslinking reaction between an alginate and a source of divalent and/or trivalent cation. The alginates useful herein are for example, sodium alginate and potassium alginate, preferably sodium alginate. The source of divalent cation includes, for example, sources of Mn, Zn, Ni, Co, Fe, Mg, Ca, Sr, Ba, Cd, Cu, Pb divalent cations, and the source of trivalent cation include, for example, sources of Fe, Cr, Al, Ga, Sc, and La trivalent cations. Among them, preferred are the source of divalent cations, and more preferred are the divalent cations selected from the group consisting of Ca divalent cation, Mg divalent cation and mixtures thereof, still more preferably Ca divalent cation.

Preferably, the alginate gel shell has a shell breakage force of from about 0.1 Newton to about 100 Newton, more preferably from about 0.5 Newton to about 50 Newton, still more preferably from about 1 Newton to about 10 Newton. The shell breakage force is measured by Motorized Compression Tester e.g. MTS Synergy/Bionix 100 or equivalent machines, with a compression plunger having a cylindrical terminal portion with a lager diameter than the diameter of capsule sample, at 23±3° C. (73±5.4° F.) and 40% RH. The shell breakage force is measured by the following method:

Place the capsule sample a test vessel; orient the capsule to ensure the largest capsule dimension lies flat on the vessel bottom surface;
Set the height of the plunger so that it rests slightly above (less than 1 mm) the capsule;
Select the Compression Rate (i.e., crosshead or ram speed) at 20 mm/min; and
Run test for each sample and record the peak force value as shell breakage force.

### Container for the Encapsulated Liquid Composition

The encapsulated liquid composition can be contained in a container for sales to consumers. One capsule of the liquid composition can be contained in one container, mainly for single dose of the composition. Alternatively, two or more capsules can be contained in one container. Such capsule or capsules can be contained in a container with or without liquid carrier to suspend the capsule or the capsules. Such liquid carriers can be anything as long as such liquid carriers have compatibility with the shell, for example, not to dissolve or break the shell. Such liquid carriers include, for example: water and other aqueous carriers such as ethanol, anhydrous liquid carrier such as oils; and same or different liquid composition from liquid composition inside of the capsule; preferably, those with preservatives.

### Method of Making

The encapsulated liquid composition of the present invention can be prepared by a method, for example, that comprising the steps:

(1) Preparing a frozen shaped unit of a liquid composition; and

(2) Forming a shell to encapsulate the frozen shaped unit.

When the shell is formed by an alginate gel, the encapsulated liquid composition of the present invention can be prepared by a method, for example, that comprising the steps:
(1) Preparing a frozen shaped unit of a liquid composition, wherein the frozen shaped unit also contains an alginate or a source of divalent and/or trivalent cation;
(2) Preparing an aqueous solution comprising an alginate or a source of divalent and/or trivalent cation which is not contained in the frozen shaped unit;
(3) Immersing the frozen shaped unit into the aqueous solution;
(4) Forming alginate gel shell by the alginate and the source of divalent and/or trivalent cation to encapsulate the frozen shaped unit.

The above method can also be used for making “capsule-in-capsule”, by containing an encapsulated 3^{rd} composition in the frozen shaped unit in the above step (1).

Frozen shaped unit herein means that the shaped unit can be fully frozen or partially frozen as long as it can keep its shape until the completion of the formation of the shell. “Partially frozen” means, frozen outside but liquid inside. For example, “80% frozen” means that the shaped unit is frozen from outside and 20% non-frozen liquid composition exists inside of the frozen shaped unit. When the shaped units are partially frozen, it is preferred to make it frozen more than 10%, more preferably more than 30%, still more preferably more than 50%, even more preferably more than 70%, furthermore preferably more than 80%. “Frozen” herein means, being frozen around 0° C. for many aqueous compositions generally, however, also means being frozen and/or being solid at any temperature under the melting point of the liquid composition. Frozen shaped unit can be prepared by any means, for example, pouring the liquid composition to a shaped mold to make it frozen in the mold, and/or preparing evenly spread sheet of the liquid composition having a certain viscosity and press the sheet by a shaped mold and make it frozen.

The aqueous solution useful herein can have a viscosity of, for example, from about 1 cp to about 600 cp, and preferably from about 10 cp to about 150 cp. The aqueous solution may further contain viscosity adjuster, agent to adjust the spend of crosslinking reaction between alginates and the source of divalent and/or trivalent cations.

Immersing can be continued preferably from about 1 sec to 24 hours, more preferably from about 1 sec to 1 hour, still more preferably from about 10 sec to 30 min, furthermore preferably from about 10 sec to 10 min, even more preferably from about 20 sec to 10 min. Before and during immersing, the aqueous solution has a temperature preferably up to about 50° C., more preferably up to about 40° C., still more preferably up to about 35° C., even more preferably up to about 30° C., and preferably from about 5° C., more preferably from about 10° C., still more preferably from about 15° C., even more preferably from about 20° C., in view of accelerating the crosslinking reaction between the cations and alginates (alginate gel shell formation) while keeping the frozen shape until the completion of the alginate gel shell formation. During immersing, it's better to avoid the frozen shaped unit contacting other frozen shaped units by, for example, using separations between such frozen shaped units.

The method may further comprise a step of injecting a 2^{nd} composition inside the shell. The 2^{nd} composition is preferably different from the liquid composition inside of the shell, more preferably is visibly distinct from the liquid composition inside of the shell. This injection step may be preferred especially when the 2^{nd} composition is:
Not frozen at conventional conditions, for example, liquid compositions containing higher amounts of alcohols having lower freezing point than that of water, liquid compositions containing higher amounts of oily compounds having lower freezing point than that of water, and also gaseous composition such as oxygen and carbon dioxide;
Containing any temperature sensitive materials such as flavor and/or scent; and
Immiscible to the liquid composition inside of the shell.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

### Liquid Composition Example 1: Aqueous Hair Shampoo Liquid Composition

Water, sodium laureth sulfate, sodium lauryl sulfate, cocamidopropyl betaine, sodium chloride, citric acid, sodium citrate, sodium xylenesulfonate, fragrance, cocamide MEA, sodium benzoate, PEG-60 almond glycerides, trisodium ethylenediamine disuccinate, tetrasodium EDTA, guar hydroxypropyltrimonium chloride, panthenol, panthenyl ethyl ether, methylchloroisothiazolinone, methylisothiazolinone

### Liquid Composition Example 2: Aqueous Hair Conditioner Liquid Composition

Water, stearyl alcohol, behentrimonium methosulfate, bis-aminopropyl dimethicone, cetyl alcohol, fragrance, benzyl alcohol, dicetyldimonium chloride, isopropyl alcohol, disodium EDTA, propylene glycol, citric acid, histidine, butylene glycol, aloe barbadensis leaf juice, argania spinose kernel oil, alcohol denatured, methylchloroisothiazolinone, methylisothiazoline, ecklonia radiata extract.

### Liquid Composition Example 3: Aqueous Hair Treatment Liquid composition

Water, Amodimethicone, Stearyl Alcohol, Cetyl Alcohol, Stearamidopropyl Dimethylamine, Fragrance, L-Glutamic Acid, Ethylene Diamine Tetraacetic Acid, Benzyl Alcohol, Panthenyl Ether, Panthenol DL 56 Solution, Methylchloroisothiazonlinone/Methylisothiazolinone Sodium Chloride, Carthamus Tinctorius (Safflower) Seed Oil, Cocos Nucifera (Coconut) Oil, Trimethylsiloxysilicate-MQ resin, Aloe Barbadensis Leaf Juice, Citric Acid Anhydrous

### Encapsulated Liquid Composition Example 1

2 wt % of calcium chloride powder is added to a hair care composition of any of the Liquid composition example 1, 2 or 3, and stirred to mix well. Separately, sodium alginate water bath at 0.5% concentration is prepared. 7 ml of the mixture of calcium chloride and the hair care composition is filled into a shaped mold such as donut-shaped, star-shaped, heart-shaped, and cloud-shaped mold, and fully frozen. The frozen shaped unit is then extracted from the mold and immersed in the sodium alginate water bath for 4 min at a room temperature to allow the formation of the alginate gel shell surrounding the shaped unit. The shaped encapsulated hair care composition is then rinsed in a water bath. The shaped encapsulated hair care composition has a shaped alginate gel shell such as donut-shaped, star-shaped, heart-shaped, and cloud-shaped shell, and contains the liquid hair care composition inside of the shell.

### Encapsulated Liquid Composition Example 2: Making of Encapsulated Hair Care Composition Using Spray Coating

7 ml of a hair care composition of any of the Liquid composition example 1, 2 or 3 is filled in a shaped mold such as donut-shaped, star-shaped, heart-shaped, and cloud-shaped mold, and fully frozen. Separately, sodium alginate water bath at 0.5% concentration is prepared. 2 wt % calcium chloride in deionized water is prepared and filled into a misting spray apparatus. The frozen shaped unit is extracted from the mold and the calcium chloride solution is sprayed on the frozen shaped unit to coat the frozen shaped unit with a very thin film of calcium chloride solution. The coated frozen shaped unit is then immersed in the sodium alginate water bath for 4 mins at a room temperature to allow the formation of the alginate gel shell surrounding the shaped unit. The shaped encapsulated hair care composition is then rinsed in a water bath. The shaped encapsulated hair care composition has a shaped alginate gel shell such as donut-shaped, star-shaped, heart-shaped, and cloud-shaped shell, and contains the liquid hair care composition inside of the shell.

### Encapsulated Liquid Composition Example 3: Making of Encapsulated Hair Care Composition Using Dip Coating

7 ml of a hair care composition of any of the Liquid composition example 1, 2 or 3 is filled in a shaped mold such as donut-shaped, star-shaped, heart-shaped, and cloud-shaped mold, and fully frozen. Separately, sodium alginate water bath at 0.5% concentration is prepared. 2 wt. % calcium chloride solution is also prepared. The frozen shaped unit is extracted from the mold and then dipped into the calcium chloride solution for 3 sec. The frozen shaped unit coated with calcium chloride is then immersed in the sodium alginate water bath for 4 mins at a room temperature to allow the formation of the alginate gel shell surrounding the shaped unit. The shaped encapsulated hair care composition is then rinsed in a water bath. The shaped encapsulated hair care composition has a shaped alginate gel shell such as donut-shaped, star-shaped, heart-shaped, and cloud-shaped shell, and contains the liquid hair care composition inside of the shell.

### Encapsulated Liquid Composition Example 4: Capsule in Capsule

### Inner Capsule

2 wt % of calcium chloride powder is added to a hair care composition of any of the Liquid composition example 1, 2 or 3, and stirred to mix well, to prepare 1^{st} hair care composition. 0.5 ml of the 1^{st} hair care composition is filled into an oval shaped mold and fully frozen. Separately, sodium alginate water bath at 0.5% concentration is prepared. The frozen shaped unit is then extracted from the mold and immersed in the sodium alginate water bath for 1-3 min at a room temperature to allow the formation of the alginate gel shell. The encapsulated hair care composition having oval shaped alginate gel shell and containing the 1^{st} liquid hair care composition inside, is then rinsed in a water bath.

### Outer Capsule

1.5 wt % of calcium chloride powder and color dye is added to a hair care composition of any of the Liquid composition example 1, 2 or 3, and stirred to mix well, to prepare 2^{nd} hair care composition. 5 ml of the 2^{nd} hair care composition is filled into a donut shaped mold with one or more pieces of the above prepared inner capsules and fully frozen. The frozen shaped unit is then extracted from the mold and immersed in the sodium alginate water bath for 5 min at a room temperature to allow the formation of the alginate gel shell. The multi-encapsulated hair care composition, having a donut shaped outer alginate gel shell capsule, and containing the 2^{nd} liquid hair composition and oval shaped inner capsules inside the donut shaped outer shell, is then rinsed in a water bath.

### Encapsulated Liquid Composition Example 5: Stripe

2 wt % of calcium chloride powder is added to a hair care composition of the Liquid composition example 2, and stirred to mix well. Blue color dye and glycerin are also added to the above composition and mixed well, to prepare 1^{st} hair care composition with a higher density. 3 ml of the 1^{st} hair care composition (having blue color and higher density) is filled into a shaped mold and frozen. Separately, 2 wt % of calcium chloride powder is added to a hair care composition of the Liquid composition example 2, and stirred to mix well, to prepare 2^{nd} hair care composition having white opaque color and lower density. 2 ml of the 2^{nd} hair care composition (having white opaque color and lighter density) is added on top of the frozen shaped 1^{st} hair care composition and place in freezer till fully frozen to prepare a frozen shaped unit. Separately, sodium alginate water bath at 0.5% concentration is prepared. The frozen shaped unit is then extracted from the mold and immersed in the sodium alginate water bath for 5 min at a room temperature to allow the formation of the alginate gel shell surrounding the shaped unit. The shaped encapsulated hair care composition is then rinsed in a water bath. The shaped encapsulated hair care composition has a shaped alginate gel shell and contains the 1^{st} and 2^{nd} liquid hair care compositions inside of the shell, and the 1^{st} and 2^{nd} liquid hair care compositions forms blue and white opaque stripes.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An encapsulated liquid composition comprising a shell and a liquid composition contained inside of the shell,
wherein the encapsulated liquid composition meets at least one of the following conditions: the encapsulated liquid composition has at least one shape other than spherical shape;
the encapsulated liquid composition contains 2 liquid compositions having different colors inside of the shell, and the 2 compositions form stripe, marble, and/or rainbow; and/or the encapsulated liquid composition contains a smaller inner capsule, wherein the smaller inner capsule is also an encapsulated liquid composition comprising a shell and the liquid composition contained inside of the shell;
wherein the shell is formed by a material selected from the group consisting of alginate gel, carrageenan gel, chitosan gel, or mixtures thereof.

2. The encapsulated liquid composition of claim 1, wherein the shell is formed by an alginate gel.

3. The encapsulated liquid composition of claim 1, wherein the liquid composition has a viscosity of from about 1 cp to about 1,000,000 cp.

4. The encapsulated liquid composition of claim 1, wherein the liquid composition has a viscosity from about 1 cp to about 500,000 cp.

5. The encapsulated liquid composition of claim 1, wherein the liquid composition has a viscosity from about 1 cp to about 200,000 cp.
